(19) United States
(12) Patent Application Publication
(10) Pub. No.: US 2006/0147516 A1
(43) Pub. Date: Jul. 6, 2006

(54) Taste masking system for alprazolam

(75) Inventors: Walid Habib, Crystal, MN (US); Derek Moe, Maple Grove, MN (US)

Correspondence Address: CIMA;LERNER, DAVID ET AL, 600 SOUTH AVENUE WEST, WESTFIELD, NJ, 07090, US

(73) Assignee: CIMA LABS INC., Eden Prairie, MN (US)

(21) Appl. No.: 11/325,038

(22) Filed: Jan. 4, 2006

Related U.S. Application Data
(60) Provisional application No. 60/641,807, filed on Jan. 6, 2005.
(60) Provisional application No. 60/642,619, filed on Jan. 10, 2005.

(51) Int. Cl. A61K 9/48; A61K 9/20

(52) U.S. Cl. 424/451; 424/464

(57) ABSTRACT

The present invention relates to taste masking system, taste masked formulations, dosage forms made from those formulations and methods of making those formulations that involve dissolving or dispersing a pH dependant polymer and alprazolam in a solvent, granulating using that material or forming layers over a solid support therewith. This can be followed with the use of an overcoating layer.

### CROSS-REFERENCE TO RELATED APPLICATIONS

This application claims the benefit of the filing date of U.S. Provisional Patent Application No. 60/641,807, filed Jan. 6, 2005 and U.S. Provisional Patent Application No. 60/642,619, filed Jan. 10, 2005, the disclosures of which are hereby incorporated herein by reference.

### BACKGROUND OF THE INVENTION

There are many advantages to changing the delivery system and format of an established drug. Some drugs which are found only in swallow tablets may be difficult for patients to swallow, particularly the elderly and small children. Developing dosage forms that can readily disintegrate in the mouth of a patient is a tremendous advantage where possible. However, it is important that such dosage forms be organoleptically pleasant, i.e., do not provide a relatively gritty sensation so as to make their ingestion unpalatable. Moreover, tablets that disintegrate in the mouth often expose the patient to the taste of the active ingredient which, not infrequently, is dreadful.

Taste masking technologies are known. However, not all taste masking technologies can work with every drug. Various taste masking technologies can, in certain instances, interfere with disintegration, provide inadequate taste masking for a given active or, as importantly, interfere with the bioavailability or pharmacokinetic properties of the drug relative to a swallow tablet. In addition, designing and producing disintegrable dosage forms that are taste masked often can increase the expense of the dosage form when compared to merely directly compressing a tablet. Often these systems require coating operations and sometimes multiple coating operations, which can be difficult and expensive. It can also require that coating apparatus be cleaned between successive coating operations or that large capital expenditures be made to purchase two or more coating apparatus.

A system which would eliminate the need for multiple coating apparatus or cleaning of multiple apparatus would be a great advantage.

### SUMMARY OF THE INVENTION

One aspect of the invention is the mixing, dissolving or dispersing of alprazolam directly with or in a taste masking material and using the resulting material as a taste masking coating. The resulting coating is also contemplated.

Another aspect of the present invention is a taste masked alprazolam formulation comprising a solid support, at least one alprazolam-containing layer which is covering at least a portion of the solid support, and at least one overcoating layer covering at least a portion of the alprazolam-containing layer. The solid support may be precoated with one or more layers over which are coated the alprazolam-containing layer(s). The alprazolam-containing layer comprises at least alprazolam, which has been discovered to be a non-plasticizing active pharmaceutical ingredient (“API”) and at least one first taste masking material. The overcoating layer can be any material but is preferably at least one of at least one second taste masking material or a material which is pH dependent and becomes soluble at a pH of about 6.5 or less. The dosage forms made using this formulation also include at least one additional ingredient generally mixed with or granulated with the taste masked particles. The additional ingredient is selected from the group consisting of binders, glidants, disintegrants, effervescent couples, colors, flavors, coatings, lubricants and carriers. In one embodiment, the first and the second taste masking materials used in the alprazolam-containing layer and the overcoating layer respectively are composed of the same material. In one embodiment, both are composed of a polymer or copolymer whose solubility is pH dependent and which becomes soluble at a pH of 6.5 or below, and more preferably an acrylic polymer or copolymer.

In another aspect of the present invention, the overcoating layer does not include alprazolam, or possibly any active pharmaceutical ingredient, aside from any which may leach in, or will be disposed at the interface between the overcoating and the alprazolam-containing layers or which is present in incidental amounts, i.e., less than 3% of the total amount of alprazolam.

In another embodiment in accordance with the present invention, the taste masked formulation can include more than one alprazolam-containing layer and/or more than one overcoating layer. When a plurality of layers are present, they may be layered in any order. For example, the solid support can be coated with an alprazolam-containing layer, which can in turn be coated with an overcoating layer, which can in turn be coated with an alprazolam-containing layer, which can in turn be coated with a second alprazolam-containing layer, and finally, a second overcoating layer. As another embodiment, the solid support can be coated with a first alprazolam-containing layer, a second alprazolam-containing layer and an overcoating layer coating at least a portion of said second alprazolam-containing layer. These alprazolam-containing layers may also contain different active pharmaceutical ingredients provided that at least one such layer comprises alprazolam. As previously stated, the solid support may be coated with one or more undercoating layers prior to the application of the alprazolam-containing layer(s).

Another aspect of the present invention is a dosage form intended to be placed in the mouth and disintegrated in the mouth before being swallowed. The dosage form in accordance with this aspect of the present invention includes a taste masked formulation as disclosed herein comprising a solid support, at least one alprazolam-containing layer covering at least a portion the solid support and at least one overcoating layer covering at least a portion of the alprazolam-containing layer. The dosage form also includes at least one additional ingredient in the form of a filler, lubricant, disintegrant, binder, glidant, effervescent couple, color, flavor, lubricant, coating and/or carrier. The resulting dosage forms are in the form of a tablet, capsule, caplet, gelcap, powder, gum, film, syrup, liquid or suspension.

In another aspect of the present invention there is provided a solid dosage form which is intended to disintegrate in the mouth of a patient, preferably in a period of two minutes or less, more preferably 90 seconds or less, and even more preferably 60 seconds or less. This dosage form is often selected from tablets, capsules, caplets, gums and films. In a particularly preferred embodiment, this rapid disintegration and taste masking is achieved without loss of bioequivalency in terms of Cₘₐₓ (with and without water) when compared to commercially available swallow tablets of alprazolam at the same dose. In another particularly preferred embodiment, this rapid disintegration in about 90 seconds or less and taste masking is achieved and which provides a release of not less than 85% of its content of alprazolam within 5 minutes when tested by a using a USP 2 paddle test as described herein in a media having a pH of 6.0.

The present invention also provides various methods of making a taste masked formulation and/or dosage form. One of these methods comprises the steps of mixing at least one non-plasticizing API comprising alprazolam with at least one first taste masking material and at least one solvent. This forms a first taste masking mixture. The solid support is then coated, at least in part, with the first taste masking mixture to form an alprazolam-containing layer and preferably allowed to dry. The solid support coated with the alprazolam-containing layer is then coated with an overcoating layer comprising a second taste masking mixture, which is itself comprised of at least one second taste masking material and at least one solvent to form an overcoating layer. Again, the first taste masking material and the second taste masking material may be the same or may be different. This process can be modified to include the application of more than one of each of the layers as described previously. The resulting taste masking materials may then be mixed with one or more additional ingredients and formed into a dosage form, such as, for example, being compressed into a tablet. Other methods which can provide dosage forms of alprazolam having comparable properties are also contemplated.

The alprazolam-containing formulations and dosage forms of the invention may be used in treating anxiety disorders, panic disorder, generalized anxiety disorder, transient symptoms of anxiety, agoraphobia, simple phobias, social phobia, posttraumatic stress disorder, obsessive-compulsive disorder, avoidant personality disorder, depression, and/or irritable bowel syndrome. The method entails administering to a subject in need of treatment an orally disintegrable dosage form comprising at least one solid support, at least one alprazolam-containing layer covering at least a portion of the at least one solid support, and at least one overcoating layer covering at least a portion the at least one alprazolam-containing layer, placing the orally disintegrable dosage form into the mouth of the subject, maintaining the dosage form in the mouth for a time which is sufficient to allow the dosage form, or portions thereof, to disintegrate and/or dissolve, and swallowing the resulting disintegrated and/or dissolved material. In a preferred embodiment, the at least one alprazolam-containing layer comprises alprazolam and at least one first taste masking material and the overcoating layer comprises at least one second taste masking material.

The dosage form preferably also includes at least one additional ingredient selected from the group consisting of binders, glidants, effervescent couples, colors, flavors, coatings, lubricants and carriers. In a particularly preferred embodiment the orally disintegrable dosage form is in the form of a compressed tablet which can disintegrate in the mouth of a patient within about 60 seconds.

In another embodiment, the process of treating patients also involves watching the patient for a period of time sufficient to ensure the disintegration of the dosage form and that the patient swallowed, thus reducing the possibility that the patient hid the dosage form in his/her mouth, only to spit it out when the health professional's back was turned. This cam be useful for psychiatric patients and anyone who is resistant to dosing compliance. It is not necessary to watch the patient in all instances. Indeed, in accordance with another aspect there is provided a method of treating a patient in need thereof by placing a tablet in accordance with the present invention in ones mouth and allowing it to at least partially disintegrate and/or dissolve followed by swallowing with saliva. In a preferred embodiment, it is placed on the top of the tongue where it dissolves/disintegrates within a few seconds prior to being swallowed.

In a particularly preferred embodiment, for anxiety disorders and transient symptoms of anxiety, patients can be initiated with a dose of 0.25 to 0.50 mg of alprazolam given three times a day. This dose may be increased to achieve a maximum therapeutic effect, at intervals of three to four days, to a maximum daily dose of 4 mg given in divided doses. Should discontinuation of dose be required, the daily dosage should be decreased by no more than 0.50 mg every three days.

For panic disorders, doses in the range of 1 to 10 mg of alprazolam daily have been used. These are provided in divided doses. In elderly patients, in patients with advanced liver disease or in patients with debilitating disease, the usual starting dose is 0.25 mg given two or three times daily. This may gradually be increased if needed and tolerated.

The dosage forms of the present invention may be swallowed with water. However, they are preferably orally disintegrable and water need not be taken. In one aspect of the present invention, mean Tₘₐₓ of the dosage forms of the present invention will occur about 15 minutes earlier when the orally disintegrable alprazolam dosage forms of the present invention are taken with water when compared to the same dosage form taken without water. There is no material change in Cₘₐₓ or area under the curve (AUC). Plasma levels remain proportional to the dose given over the dosage range of 0.30 to 3.0 mg with peak levels at 8.0 to 37 ng/ml being observed. Moreover, in one embodiment, notwithstanding the change from a swallow tablet to an orally disintegrable tablet, food decreases the mean Cₘₐₓ by about 25% and increases the mean Tₘₐₓ by two hours, from about 2.2 hours to about 4.4 hours after ingestion of a high fat meal. Food does not affect the area under the curve.

The present invention provides numerous advantages. Eudragit E-100, for example, can be dissolved or dispersed in a number of solvents such as alcohol or water. This allows one to dissolve or suspend drugs which are, for example, water insoluble or incompatible. Spraying the resulting mixture onto the surface of a solid support helps reduce the overall exposed drug surface area, assisting in taste masking by reducing the degree of exposure. In addition, the Eudragit E-100 is capable of providing taste masking in and of itself. This further enhances the overall taste masking achieved in accordance with the present invention.

Not only does the Eudragit E-100 used in this type of formulation provide superior taste masking, it acts as a good binder and is relatively non-tacky and easily processed. This improves workability, content uniformity and the like. Moreover, because, in this embodiment, the taste masking coating used in the overcoating layer and the taste masking coating contained in the API layer can be made from the same material, one need not use a second coating apparatus or necessarily interrupt the process to clean and reconfigure for a coating using a separate material. Indeed, one can, without significant interruption, and even without drying, stop the feed of the alprazolam-containing material and begin feeding in the overcoating material. This can save considerable processing time without sacrificing performance.

In still another embodiment, there is provided an orally disintegrable tablet that can disintegrate in the mouth within about 90 seconds or less and which provides a release of not more than about 45% of its content of alprazolam within about 5 minutes when tested by a USP 2 apparatus using a USP 2 paddle test as described herein in a media having a pH of about 6.8. In still another embodiment, there is provided an orally disintegrable tablet that can disintegrate in the mouth within about 90 seconds or less and which provides a release of not less than about 85% of its content of alprazolam within about 5 minutes when tested by a using a USP 2 paddle test as described herein in a media having a pH of about 6.0. In another embodiment, it will meet both of the above standards.

In another embodiment, the alprazolam-containing layer material, e.g., the combination of the alprazolam and the first taste masking material, can be used as a granulation binder. The resulting granulate can be coated with the one or more overcoating layers directly or can first be coated with one or more alprazolam-containing layers prior to application of one or more overcoating layers.

In another embodiment, there is provided an alprazolam-containing ODT tablet that provides adequate taste masking as measured by a bitterness analysis.

In another embodiment, there is provided a taste masked formulation comprising: a solid support, at least one alprazolam-containing layer covering at least a portion of the solid support and at least one overcoating layer covering at least a portion of the alprazolam-containing layer. The alprazolam-containing layer comprises alprazolam and at least one first taste masking material and the overcoating layer comprises at least one second taste masking material. In one aspect of this embodiment, the first and/or the second taste masking materials are pH dependent and become soluble at a pH of about 6.5 or less.

In yet another embodiment, there is provided a pharmaceutical dosage form comprising: alprazolam and at least a first taste masking material. The dosage form disintegrates in the mouth within about 90 seconds or less and provides a release of not more than 45% of its content of alprazolam within 5 minutes when tested by a USP 2 paddle test in a media having a pH of 6.8.

In still another embodiment, there is provided a pharmaceutical dosage form comprising: alprazolam and at least a first taste masking material. The dosage form disintegrates in the mouth within about 90 seconds or less and provides a release of not less than 85% of its content of alprazolam within 5 minutes when tested by a USP 2 paddle test in a media having a pH of 6.0.

And in still another embodiment, there is provided a pharmaceutical dosage form comprising: alprazolam and at least a first taste masking material. The dosage form disintegrates in the mouth within about 90 seconds or less and provides a release of not more than 45% of its content of alprazolam within 5 minutes when tested by a USP 2 paddle test in a media having a pH of 6.8 and provides a release of not less than 85% of its content of alprazolam within 5 minutes when tested by a USP 2 paddle test in a media having a pH of 6.0.

In another embodiment, there is provided a pharmaceutical dosage form comprising: alprazolam and at least a first taste masking material. The orally disintegrable dosage form can disintegrate in the mouth within about 90 seconds or less and provides a release of not more than 45% of its content of alprazolam within 5 minutes when tested by a USP 2 paddle test in a media having a pH of 6.8 and/or provides a release of not less than 85% of its content of alprazolam within 5 minutes when tested by a USP 2 paddle test in a media having a pH of 6.0. The at least one first taste masking material and is pH dependent and become soluble at a pH of about 6.5 or less.

In yet another embodiment, there is provided a pharmaceutical dosage form comprising: a solid support, at least one alprazolam-containing layer covering at least a portion of the solid support, at least one overcoating layer covering at least a portion of the alprazolam-containing layer. The dosage form also includes at least one additional ingredient. The alprazolam-containing layer comprises alprazolam and at least one first taste masking material and the overcoating layer comprises at least one second taste masking material. The first and the second taste masking materials are pH dependent and become soluble at a pH of about 6.5 or less and wherein said orally disintegrable dosage form can disintegrate in the mouth within about 90 seconds or less and provides either a release of not more than 45% of its content of alprazolam within 5 minutes when tested by a USP 2 paddle test in a media having a pH of 6.8, a release of not less than 85% of its content of alprazolam within 5 minutes when tested by a USP 2 paddle test in a media having a pH of 6.0, or both.

### DETAILED DESCRIPTION

Throughout the entire specification, including the claims, the word “comprise” and variations of the word, such as “comprising” and “comprises,” as well as “have,” “having,” “includes,” “include” and “including,” and variations thereof, means that the named steps, elements or materials to which it refers are essential, but other steps, elements or materials may be added and still form a construct with the scope of the claim or disclosure. When recited in describing the invention and in a claim, it means that the invention and what is claimed is considered to what follows and potentially more. These terms, particularly when applied to claims, are inclusive or open-ended and do not exclude additional, unrecited elements or methods steps. The term “between” as used in connection with a range includes the endpoints unless the context suggests otherwise. All references to testing is at room temperature (20-25° C.) unless otherwise specified and all references to temperature are in degrees centigrade unless otherwise specified.

In the present context, “consisting essentially of” is meant to exclude any excipient or combination of excipients or, as appropriate, any amount of any excipient or combination of excipients, as well as any pH adjusting substance or any amount of pH adjusting substance that would alter the basic and novel characteristics of the invention.

A solid support in accordance with the present invention can be composed of any material useful for layering in accordance with this and other conventional pharmaceutical applications. These can include, without limitation, particles, crystals, granulates, capsules, microparticles, microgranules, microcrystals or microcapsules. Particles, granules and crystals have their traditional meaning. “Capsule” in accordance with the present invention includes generally hollow, spherical vessels such as liposomes, micelles and the like. These may be dried. Solid supports can be composed of any number of materials or mixtures thereof including particles created from one or more of the taste masking materials, polymers, solid dicalcium phosphate and the like. However, in a preferred embodiment, the solid supports are made of a sugar. Sugar in accordance with the present invention generally includes other forms of carbohydrate such as, for example, sugars, sugar alcohols, ketoses, saccharides, polysaccharides, oligosaccharides and the like, as well as celluloses and modified celluloses. These include, without limitation, sucrose, mannitol (spray dried and granular) lactose, and microcrystalline cellulose. Most preferred in accordance with the present invention are sucrose and microcrystalline cellulose. Useful sucrose spheres are available from Paulaur corporation, 105 Melrich Road, Cranbury, N.J. 08512. Useful microcrystalline spheres are sold by Asahi Kasei Chemicals Corp, with the following address: Hibiya-Mitsui Building 1-2 Yurakucho 1-chome, Chiyoda-ku, Tokyo 100-8440 Japan under the designation CELPHERES.

The size of the solid support can vary considerably with, amongst other things, the application, volume of the solid support that will be used in the formulation, the type of dosage form in which it will be included, and the thicknesses of the layers that will coat it. Solid supports that are too small can be difficult to coat. Solid supports that are too large can be difficult to work with, can affect content uniformity and can provide an unpleasant organoleptic sensation in the mouth. Of course, the larger the particle size, the smaller the surface area of the alprazolam that will be provided in the mouth thus reducing the potential exposure to the taste buds and other sensory organs within the mouth, further enhancing taste masking. Size may also vary depending upon the use of undercoatings. Thus an undercoating layer of E-100 could be applied to the solid support prior to application of an alprazolam-containing layer.

In accordance with the present invention, the solid support size is preferably between about 10 microns and about 1,000 microns, more preferably between about 20 microns and 600 microns. This means that at least about 90% of the solid support, by weight, fall within these ranges based on sieving. In a more preferred embodiment, the solid support will be predominantly solid support falling within a 60 to 80 mesh screen cut. More particularly, the amount by weight greater than 300 μm is about 0%, the amount by weight greater than 250 μm is less than about 10%, the amount in between about 180 and about 250 μm is about 90% or more, and the amount by weight less than 180 μm is about 10% or less.

A 45-60 mesh screen cut with similar percentages may also be preferred. Again, about 90% of the particles should be between about 250 microns and about 350 microns. This is measured as before. “Micro” in the context of solid supports means a solid support having a particle size of below about 50 microns. Preferably the solid support is substantially spherical although the particle dimensions can vary and can be, without limitation, elliptical, generally egg-shaped, rod-shaped, regular and/or irregularly shaped.

Covering at least a portion of the solid support is at least one alprazolam-containing layer. By “covering at least a portion” in context of the alprazolam-containing layer, it is understood that the complete surface area of each particle need not be covered. Indeed, while the efficiency of the system is improved considerably by the use of substantially complete and uniform coating, thus reducing the number of solid support particles necessary to deliver a given amount of API, it is not required that the coating of the alprazolam-containing material cover even a majority of the particles of solid support or a majority of the surface of the solid support. Preferably, however, the alprazolam-containing layer covers substantially all of the solid support to which it is applied (it is possible to mix some coated and uncoated solid support if desired). By “substantially all” it is understood that, generally speaking, at least about 85% by weight of the coating material (the API and first taste masking material) used is actually coated onto the solid support. Thus, at least about 85% of the API coating layer applied (API and first taste masking material) actually coats the solid support. Relatively little, therefore, is wasted.

The alprazolam-containing layer, and indeed the overcoating layer as well, can be applied by any normal process such as use of a Wurster fluidized bed where the coating material enters from the bottom of the reactor. When this process was used, it was found that 85% or more by weight of the alprazolam-containing coating could be applied to the solid support. For example, if 1 kilogram of coating were prepared and used in the process, at least 850 grams would actually end up on the solid support particles. The amount of alprazolam-containing coating material can also be calculated based on the weight gain of the solid support. Thus the amount of coating can result in a weight gain of between about 0.1 and about 300%, more preferably between about 1.0 and about 200% by weight of the alprazolam-containing coating relative to the weight of the solid support. This is based on the total amount of the alprazolam and the first taste masking material and does not include solvent or other coating additives.

The at least one first taste masking material useful in accordance with the present invention generally includes any natural or synthetic polymer including: acrylic polymers, modified celluloses, and the like, which are pH dependant materials that become soluble at a pH of 5.5-6.0 or below. These polymers and copolymers should preferably be pharmacologically acceptable, capable of providing appropriate release and effective taste masking while still being convenient to process. These include, for example, amino alkyl acrylate copolymers such as, for example, copolymers of methylmethacrylate, butylmethacrylate and dimethylaminoethyl methacrylate. See European Pharmacopoeia 4.4 (April/2003:1975) at 3385. In one particularly preferred embodiment, the copolymer has a relative molecular mass of about 150,000 and a ratio of dimethylaminoethyl methacrylate groups to butylmethacrylate groups and methylmethacrylate groups of about 2:1:1 and the content of the dimethylaminoethyl groups is about 20.8% to 25.5% based on the amount of dry substances present.

A particularly preferred material can be obtained under the mark Eudragit E-100, which can be used in normal form or in micronized Eudragit E-100 and mixtures thereof. Eudragit is a trademark of Rohm GmbH, Chemische Fabrik, Kirschenallee, D-64293, Darmstadt, Germany for a group of acrylic polymers.

These materials are generally solid at room temperature. However, they may be applied to the solid support and mixed with the alprazolam by being dissolved, suspended, emulsified, dispersed or the like in a solvent or solvent system. Preferred solvents in accordance with the present invention include those capable of substantially dissolving or dispersing Eudragit E-100 such as water, normal C₁-C₅ alcohol, branched C₁-C₅ alcohol, denatured C₁-C₅ alcohol, and low molecular weight ketones such as acetone and MEK. Ethanols, including (SDA-3A) and denatured ethanol are most preferred.

The active pharmaceutical ingredient useful in accordance with the present invention is alprazolam which has been found to be “non-plasticizing.” This is a pharmaceutically active material that is relatively non-tacky and generally will remain relatively non-tacky so as to render coated solid supports workable when combined with the first taste masking material, whether or not up to about 25% by weight of a conventional anti-tack agent, such as talc or magnesium stearate is added. A “plasticizing” active pharmaceutical ingredient cannot meet this requirement. They will be relatively tacky and unworkable in an active pharmaceutical ingredient-containing layer, even with 25% of an anti-tacking agent. If APIs are used in addition to alprazolam, they would preferably be non-plasticizing as well.

Indeed, it was found that when certain drugs were mixed with Eudragit E-100 and applied to sugar spheres, the result was a gummy, sticky mess that, when dried, could not be properly processed into uniform particles of the desired composition, nature and properties. The workability of this material was poor. Often plasticizing active pharmaceutical ingredients will not permit overcoating without any interruption as is the case with the preferred non-plasticizing active pharmaceutical ingredients of the invention.

Some drugs or active pharmaceutical ingredients such as, for example, alprazolam, although sparingly soluble at working concentrations in ethanol, was found to coat quite nicely and allowed application of the overcoating layer in the same equipment without interruption other than that necessary to change the feed of coating material. The resulting particles were discreet, non-tacky and exceptionally workable. By working with various materials, and without wishing to be bound by a particular theory, it was determined that certain drugs react and/or interact with the polymer materials in the taste masking coating material, changing their individual characters rendering the material more tacky. The present invention intends to encompass only those active pharmaceutical ingredients that would not so adversely affect workability so as to prevent their effective use in forming discrete, preferably free flowing, well coated, well characterized solid supports, which may be further coated. Active pharmaceutical ingredients that may be used in addition to alprazolam in accordance with the present invention may include, without limitation, analgesics, anti-inflammatories, antipyretics, antibiotics, antimicrobials, anxiolytics, laxatives, anorexics, antihistamines, antidepressants, antiasthmatics, antidiuretics, antiflatuents, antimigraine agents, antispasmodics, sedatives, antihyperactives, antihypertensives, tranquilizers, decongestants, beta blockers, peptides, proteins, oligonucleotides and other substances of biological origin, and combinations thereof. Also contemplated are the drugs and pharmaceutically active ingredients described in Mantelle, U.S. Pat. No. 5,234,957, in columns 18 through 21. That text of Mantelle is hereby incorporated by reference. Most preferably, these other active pharmaceutical ingredients are non-plasticizing as described herein.

The amount of solvent used in forming the alprazolam-containing coating will depend on, among other things, the taste masking coating material used. Moreover, more solvent may be needed to achieve dissolution than dispersion, for example. However, since the solvent is generally removed by drying, it should not make up an appreciable portion of the final product (generally less than 5% total, preferably less than 3% and more preferably less than 1% total) and therefore, the amount of solvent is not generally considered in describing the overall composition of the alprazolam-containing layer or for that matter, the overcoating layer. The amount of the alprazolam and/or total drug including alprazolam in the alprazolam-containing layer can vary from between about 0.1% to about 90% by weight of said alprazolam-containing layer. More preferably the amount ranges from between about 1% to about 75% by weight. The alprazolam-containing layer may also include anti-tack agents such as magnesium stearate or talk and copolymers such as HPMC, EC, HPC and PVP in an amount of up to about 25% by weight of that coating.

The amount of alprazolam used in each dosage form in accordance with the present invention will vary. However, generally, the taste masked dosage forms in accordance with the present invention will provide a dose of API between about 0.10 micrograms and about 2 grams, preferably between about 0.50 micrograms and about 1 gram per dosage form (e.g., tablet, teaspoonful, etc.), most preferably between about 10 micrograms to about 0.5 grams. Particularly preferred are dosage forms including 0.25, 0.50, 1.0 and 2.0 mg each.

The overcoating layer comprises at least a second taste masking material. This taste masking material, like the first taste masking material, can be any polymeric material that can effectively taste mask the alprazolam and becomes soluble at a pH of about 5.5 to about 6.0 or below as previously described. More preferably, the second taste masking material is selected from the same group of materials previously identified for the first taste masking material including Eudragit E-100. In a particularly preferred embodiment, the second taste masking material is identical to the first taste masking material. Thus, both the alprazolam-containing layer and the overcoating layer may be made from the same polymeric material.

The overcoating layer covers at least a portion of the alprazolam-containing layer. “Covering at least a portion of” in the context of the overcoating layer means that an effective portion of the surface area of the solid support coated with the alprazolam-containing layer is covered. Preferably, this effectively provides taste masking. The adequacy and completeness of the coating can be measured by weight increase as suggested herein so long as the resulting material provide adequate taste masking as measured by dissolution of 45% or less at 5 min. at pH 6.8 as described herein. Indeed, the fact that the release is less than 45% under these conditions alone suggest the overall adequacy of both the alprazolam containing coating and the overcoating. Preferably, “substantially all” of the alprazolam-containing layer is coated with the overcoating which, in the context of the overcoating layer, means that at least about 85% of the coating material used, the second taste masking material (without considering any solvent or additive), actually coats the alprazolam-containing-layer-coated solid support. The amount of overcoating material can also be calculated based on the weight gain of the solid support which has been coated with the first taste-masking coating. Thus the amount of coating can result in a weight gain of between about 0.1 and about 300%, more preferably between about 1.0 and about 200% by weight of the overcoating relative to the weight of the solid support and first taste-masking coating. This is based on the total amount of the overcoating material and does not include solvent or other coating additives. In a particularly preferred embodiment, the amount of each coating layer ranges from about 1 to about 50% based on the weight of the solid support or solid support and first taste-masking layer as appropriate. Indeed, in one embodiment, the weight increase of the first taste masking layer was about 12.5% and the weight increase of the overcoating layer was about 25%.

In the alternative, the total amount of all coating materials used can range from about 0.2 to about 1000% by weight based on the initial weight of the solid support, more preferably, from about 1 to about 600% and even more preferably from about 2 to about 400%.

It is noted that while the overcoating layer is preferably composed of a second taste masking material as described herein, it need not be. Other coatings including but not limited to those which become soluble at a pH of about 6.5 or below are contemplated to the extent that they meet the overall objectives of the invention. In another embodiment, the combination of the alprazolam-containing layer and the overcoating layer are able to provide taste masking as measured by drug release under specified conditions. Specifically, solid dosage forms made from the taste masked formulations of the invention can be tested using a USP 2 paddle method (50 r.p.m.) in 500 mL of phosphate buffered water at pH of 6.8 and 37° C. This is referred to herein as the “USP 2 paddle test.” Generally, if amount of drug released under these conditions after five minutes is 45% or less, suitable taste masking has been achieved. See Tables 1 and 2 below. Preferably release is less than 35% in five minutes. With particularly bad tasting drugs, the drug release after five minutes should be no more than about 30% weight of the alprazolam. Indeed, in some embodiments, it may be necessary or desirable that the percent release in 5 minutes at pH 6.8 is no more than about 25% by weight and in still another embodiment, not more than about 20% by weight.

TABLE 1
% Release of Alprazolam 1/10th Scale
Registration* Batch Tablets in pH 6.8 Dissolution Medium

Sample ID

| | | | |
|---|---|---|---|
| 0.25 mg¹ | 0.5 mg² | 1 mg³ | 2 mg⁴ |

| | |
|---|---|
| Time Point (min) | Average % Released (n = 3) |

| | | | | |
|---|---|---|---|---|
| 2 | NT | 8 | 12 | NT |
| 5 | 20 | 19 | 19 | 15 |
| 10 | 41 | 40 | 41 | 35 |
| 15 | 55 | 54 | 52 | 45 |
| 30 | 74 | 73 | 67 | 62 |

NT = not tested
*Registration Batch Tablets tested were stored for 27M @ ambient storage conditions

TABLE 2
% Release of Alprazolam Full Scale Batch
Tablets in pH 6.8 Dissolution Medium

| | Sample ID | | | | |
|---|---|---|---|---|---|
| | 0.25 mg¹ | 0.5 mg² | 1 mg³ | 2 mg⁴ | 2 mg⁴ |
| Time Point (min) | Average % Released (n = 3) | | | | |
| 2 | 5 | 11 | 6 | 3 | 5 |
| 5 | 14 | 19 | 14 | 7 | 12 |
| 10 | 35 | 35 | 28 | 16 | 26 |
| 15 | 52 | 49 | 41 | 24 | 39 |
| 30 | 75 | 70 | 63 | 40 | 59 |

¹0.25 mg tablets had a formulation such as that described generally in example 2.
²0.50 mg tablets had a formulation such as that described generally in example 3.
³1.0 mg tablets had a formulation such as that described generally in example 4.
⁴2.0 mg tablets had a formulation such as that described generally in example 5.

All dissolution samples were prepared using plastic syringes, pretreated PE filter tips, and 13-mm diameter, 0.45 μm, GHP syringe filters. Approximately 2 mL of the sample aliquot was filtered through the GHP syringe filter prior to collection in the HPLC vial.

TABLE 3
% Release of 0.25 mg Alprazolam Tablets
(10 kg batch size using different mesh size coated AL)
(pH 6.8 Medium)¹

| | | 0.25 mg | 0.25 mg | 0.25 mg |
|---|---|---|---|---|
| | | (Coated AL | (Coated AL | (Coated AL |
| Time | 0.25 mg | passed through | passed through | passed through |
| Point | (Control) | a 40 mesh) | a 45 mesh) | a 50 mesh) |
| (min) | Average % Released (n = 3) | | | |
| 2 | 7 | 8 | 8 | 11 |
| 5 | 16 | 20 | 19 | 23 |
| 10 | 38 | 41 | 43 | 47 |
| 15 | 54 | 58 | 60 | 63 |

TABLE 4
% Release of Alprazolam 1/10^{th} Scale Registration Tablets
(pH 6.8 Medium)

| | | | | |
|---|---|---|---|---|
| Time Point | 0.25 mg¹ | 0.5 mg² | 1 mg³ | 2 mg⁴ |

| | |
|---|---|
| (min) | Average % Released (n = 3) |

| | | | | |
|---|---|---|---|---|
| 2 | NT | 8 | 12 | NT |
| 5 | 20 | 19 | 19 | 15 |
| 10 | 41 | 40 | 41 | 35 |
| 15 | 55 | 54 | 52 | 45 |
| 30 | 74 | 73 | 67 | 62 |

NT = Not Tested

TABLE 5
% Release of Alprazolam Full Scale
Alprazelam ™ Tablets
(Validation and Commercial)
(pH 6.8 Medium)

| Time Point | 0.25 mg¹ | 0.5 mg² | 1 mg³ | 2 mg⁴ | 2 mg⁴ |
|---|---|---|---|---|---|
| (min) | Average % Released (n = 3) | | | | |
| 2 | 5 | 11 | 6 | 3 | 5 |
| 5 | 14 | 19 | 14 | 7 | 12 |
| 10 | 35 | 35 | 28 | 16 | 26 |
| 15 | 52 | 49 | 41 | 24 | 39 |
| 30 | 75 | 70 | 63 | 40 | 59 |

Dissolution Results in pH 6.8 Medium

TABLE 6
% Release of Commercial Xanax Tablets
(pH 6.8 Medium)

| | | |
|---|---|---|
| | 0.25 mg Xanax, Lot# | 2 mg Xanax, Lot# |
| Time Point | 23DYS | 92HKB |

| | |
|---|---|
| (min) | Average % Released (n = 3) |

| | | |
|---|---|---|
| 2 | 56 | 25 |
| 5 | 79 | 72 |
| 10 | 91 | 93 |
| 15 | 94 | 96 |

Note that registration tablets tested were stored for 27M @ ambient storage conditions. As aptly demonstrated by tables 3-6, the release of alprazolam from tablets of the invention at pH. 6.8 at 5 minutes is less that 45%, indeed, less than 30%. In contrast, commercially available swallow tablets of alprazolam sold under the trademark XANAX tested under substantially the same conditions showed a release of greater than 72% at 5 minutes This suggests that the commercial tablets may insufficiently taste masked.

In one preferred embodiment, both the alprazolam-containing layer and the overcoating layer include at least one polymeric material that is common to both. In a most preferred embodiment, the at least one second taste masking material used in the overcoating layer is identical to the at least one first taste masking material used in the alprazolam-containing layer. In one embodiment, both are Eudragit E-100.

Either layer may be made of a mixture of taste masking materials where none, some or all of the material used in each layer are the same or different. The taste masked formulations of the invention can be produced by dissolving or dispersing the second taste masking material in at least one solvent, as was previously described in the context of the first taste masking mixture to form a second taste masking mixture. The at least one solvent is preferably the same as those previously described in connection with the alprazolam-containing layer. This material is then coated on top of the at least one first alprazolam-containing layer to form an overcoating layer. Again, preferably, after applied, the at least one solvent would be removed, preferably by drying. In one preferred embodiment, however, there is no need to dry the alprazolam-containing layer before application of the overcoating layer. Indeed, most preferably, there is no need to interrupt the coating process, or even clean or change apparatus. One need only discontinue application of the first taste masking material and API and may, immediately if desired, begin application of the second taste masking material.

Dosage forms in accordance with the present invention are preferably solid dosage forms which are designed to disintegrate rapidly in the mouth of a patient once placed in his mouth. By “rapidly,” it is understood that these dosage forms preferably disintegrate in the mouth in less than 120 seconds, more preferably 90 second or less, even more preferably 60 seconds or less, and most preferably 45 seconds. “Disintegration” in this context refers to the break up of the tablet into constituent particles. Note that the taste masked formulation (the taste masked beads or granulate) in accordance with the present invention should not dissolve or disintegrate, as individual units, to any discernable degree (as established in a bitterness test or otherwise) during the time that they are within the mouth. That is to say while the dosage form may disintegrate and/or portions of it may dissolve in the mouth, the taste masked particles should largely remain intact while in the mouth. It is also possible that some or all of the additional ingredients contained within the dosage form will disintegrate and/or dissolve within the period of time prescribed. Dissolution in accordance with the present invention means that the material will actually be soluble in saliva as opposed to merely breaking down to constituent particles.

Solid dosage forms in accordance with the present invention include tablets, capsules, caplets, gels and films, as well as powders. While orally disintegrable solid dosage forms are preferred in accordance with the present invention, the present invention also encompasses the use of the taste masked formulations in accordance with the present invention in liquids, syrups and suspensions. Of course, to do so, at least the overcoating layer must be insoluble in the liquid carrier used for the formulation.

The dosage forms may include as additional ingredients or excipients glidants, fillers, lubricants, binders, sweeteners, disintegrants, flavoring and coloring components. Any conventional sweetener or flavoring component may be used. Combinations of sweeteners, flavoring components, or sweeteners and flavoring components may likewise be used.

An effervescent couple, alone or in combination with other ingredients may be used to improve the disintegration profile and the organoleptic properties of the dosage form. Effervescent couples are made from a reaction of a soluble acid source and a metal carbonate or bicarbonate. The acid sources or acid may be any which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations of the present invention were intended to be dissolved in a glass of water. Acid anhydrides and acid salts of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate. These effervescent couples may be provided in an amount of between about 3% and about 25% by weight of the dosage form.

In addition to the effervescence-producing agents, a dosage form according to the present invention may also include, instead of or in addition thereto, suitable non-effervescent disintegration agents. Non-limiting examples of non-effervescent disintegration agents include: microcrystalline, cellulose, croscaramellose sodium, crospovidone, starches, corn starch, potato starch and modified starches thereof, clays, such as bentonite, alginates, gums such as agar, guar, locust bean, karaya, pecitin and tragacanth. These non-effervescent disintegrants may comprise up to about 20 weight percent and preferably between about 2% and about 10% of the total weight of the dosage form.

Examples of binders which can be used include but are not limited to acacia, tragacanth, gelatin, starch, cellulose materials such as methyl cellulose, microcrystalline cellulose and sodium carboxy methyl cellulose, alginic acids and salts thereof, magnesium aluminum silicate, polyethylene glycol, PVP, guar gum, polysaccharide acids, bentonites, sugars, invert sugars and the like. Binders may be used in an amount of up to 60 weight percent and preferably about 10 to about 40 weight percent of the total dosage form.

Coloring agents may include but are not limited to titanium dioxide, and dyes suitable for food such as those known as F.D.& C. dyes and natural coloring agents such as grape skin extract, beet red powder, beta-carotene, annato, carmine, turmeric, paprika, etc. The amount of coloring used may range from about 0.1 to about 3.5 weight percent of the total dosage form.

Examples of glidants include but are not limited to silicon dioxide, talc, calcium stearate, magnesium stearate, stearowet C, zinc stearate, calcium silicate, starch, pregelatinized starch, magnesium lauryl sulfate, magnesium carbonate, magnesium oxide, and others These may be used in an amount of between about 0.1 and about 5% by weight of the dosage form.

Diluents or Fillers include, but are not limited to spray-dried monohydrate or anhydrous lactose, sucrose, dextrose, mannitol, sugar alcohols, sorbitol, starch, cellulose (e.g., microcrystalline cellulose) dihydrated or anhydrous dibasic calcium phosphate, tricalcium phosphate, maltodextrins, calcium carbonate, calcium sulfate and others. These may be used in an amount of between about 10 and about 90% by weight of the dosage form.

Examples of carriers include liquid sugar, syrup, water and the like. Examples of disintegrants include but are not limited to starches, clays, microcrystalline celluloses, celluloses, algins, gums or cross linked polymers, PVP-XL, sodium starch glycolate and croscarmellose sodium, and effervescent agents. Effervescent agents include but are not limited to: the acid sources or acid may be any which are safe for human consumption and may generally include food acids, acid anhydrides and acid salts. Food acids include citric acid, tartaric acid, malic acid, fumaric acid, adipic acid, and succinic acids etc. Acid anhydrides and acid of the above described acids may also be used. Acid salts may include sodium, dihydrogen phosphate, disodium dihydrogen pyrophosphate, acid citrate salts and sodium acid sulfite. Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors may be present in an amount ranging from about 0.05% to about 3% by weight based upon the weight of the dosage form.

Lubricants may also be used. Hydrophobic lubricants are preferred. Hydrophobic Lubricants include, without limitation, calcium stearate, magnesium stearate, zinc stearate, stearic acid, stearowet C, mineral oil, vegetable oil, glyceryl behenate, sodium stearyl fumarate, talc, starch, and others. Hydrophilic lubricants include, without limitation, sodium benzoate, sodium chloride, sodium lauryl sulfate, magnesium lauryl sulfate, polyethylene glycol, and others Magnesium stearate is preferred. These may be used in an amount of between about 0.5% and about 5% by weight, more preferably 0.5% to about 2.5% by weight of the dosage form. If desired the dosage form may also contain minor amounts of nontoxic substances such as wetting or emulsifying agents, pH buffering agents and the like, for example, sodium acetate, sorbitan monolaurate, triethanolamine, sodium acetate, triethanolamine oleate, sodium lauryl sulfate, dioctyl sodium sulfosuccinate, polyoxyethylene sorbitan fatty acid esters.

The dosage forms in accordance with the present invention preferably have a hardness of at least about 5 Newtons and are designed to disintegrate rapidly in the mouth of a patient in less than about 2 minutes, preferably 90 seconds, to thereby liberate the taste masked formulations of the invention. Preferably the dosage form will disintegrate in less than 60 seconds and even more preferably 45 seconds. This measure of hardness is based on the use of small tablets of less than about 0.25 inches in diameter. A hardness of at least about 10 Newtons is preferred for larger tablets. Most preferably, however, the dosage forms in accordance with the present invention have a hardness of between about 10 and about 150 Newtons and, more preferably, between about 10 and about 120 Newtons. Proportionate hardnesses are expected for tablets of various sizes.

When the dosage forms in accordance with the present invention are tablets, they are preferably sufficiently robust that they can be tabletted using conventional tabletting and handling equipment, as well as packaged in traditional multi-tablet bottles. See U.S. Pat. No. 6,024,981. These tablets preferably have a hardness of at least about 15 Newtons and most preferably a friability of less than 2% when measured by U.S.P., more preferably less than 1% when measured by U.S.P. Most preferably the tablets in accordance with this aspect of the invention have a hardness of between about 15 and about 100 Newtons and a friability of 1 or less when measured by U.S.P. See again U.S. Pat. No. 6,024,981.

Tablets can either be manufactured by direct compression, wet granulation, dry granulation or any other tablet manufacturing technique. See, e.g., U.S. Pat. Nos. 5,178,878 and 5,223,264, which are incorporated by reference herein. Other dosage forms in accordance with the present invention can be made in their traditional manner using the taste masking formulation as a part of their components. Liquid forms can be made by dispersing, suspending, emulsifying, or forming a colloid of the particles of the taste mask formulation of the present invention in one or more conventional delivery vehicles.

Thus in one preferred embodiment, at least the overcoating layer in accordance with the present invention can be produced from Eudragit E-100, or other materials of similar properties described herein,

Alprazolam is useful for treating anxiety disorders, panic disorder, generalized anxiety disorder, transient symptoms of anxiety, agoraphobia, simple phobias, social phobia, posttraumatic stress disorder, obsessive-compulsive disorder, avoidant personality disorder, depression and irritable bowel syndrome. Thus, the present invention includes a method of treating anxiety disorders, panic disorder, generalized anxiety disorder, transient symptoms of anxiety, agoraphobia, simple phobias, social phobia, posttraumatic stress disorder, obsessive-compulsive disorder, avoidant personality disorder, depression and irritable bowel syndrome in a subject in need of such treatment. This method includes the following steps: administering to the subject an orally disintegrable tablet comprising, at least one solid support, at least one alprazolam-containing layer covering the at least one solid support, and at least one overcoating layer covering at least a portion of the at least one alprazolam-containing layer, placing said orally disintegrable tablet into the mouth of the subject, maintaining the tablet in the mouth of the subject for a time which is sufficient to allow the tablet to disintegrate and/or dissolve, and swallowing the resulting disintegrated and/or dissolved tablet. It is preferred that the at least one alprazolam-containing layer comprise alprazolam and at least one first taste masking material and that the at least one overcoating layer comprising at least one second taste masking material. The dosage form may also include at least one additional ingredient selected from the group consisting of binders, glidants, effervescent couples, color, flavors, coatings, lubricants, and carriers. It is also preferred that the orally disintegrable dosage form be in the form of a compressed tablet which can disintegrate in the mouth of a patient within about 60 seconds. In a preferred embodiment, the tablet is placed on top of the tongue and allowed to disintegrate/dissolve and the than swallowed. The patient may be watch for a time sufficient to ensure that the tablet has been dissolved and swallowed.

In one particular embodiment, for anxiety disorders and transient symptoms of anxiety, patients can be initiated with a dose of 0.25 to 0.50 mg of alprazolam given three times a day. This dose may be increased to achieve a maximum therapeutic effect, at intervals of three to four days, to a maximum daily dose of 4 mg given in divided doses. Should discontinuation of dose be required, the daily dosage should be decreased by no more than 0.50 mg every three days.

For panic disorders, doses in the range of 1 to 10 mg of alprazolam daily have been used. These are provided in divided doses in elderly patients, in patients with advanced liver disease or in patients with debilitating disease, the usual starting dose is 0.25 mg given two or three times daily. This may gradually be increased if needed and tolerated.

The dosage forms of the present invention may be swallowed with water. However, they are preferably orally disintegrable and water need not be taken. In one aspect of the present invention, mean Tₘₐₓ of the dosage forms of the present invention will occur about 15 minutes earlier when the orally disintegrable alprazolam dosage forms of the present invention are taken with water when compared to the same dosage forms taken without water. There is no material change in Cₘₐₓ or area under the curve. Plasma levels remain proportional to the dose given over the dosage range of 0.30 to 3.0 mg with peak levels at 8.0 to 37 ng/ml being observed. Moreover, in one embodiment, notwithstanding the change from a swallow tablet to an orally disintegrable dosage form, food decreases the mean Cₘₐₓ by about 25% and increases the mean Tₘₐₓ by two hours, from about 2.2 hours to about 4.4 hours after ingestion of a high fat meal. Food does not affect the area under the curve.

In still another embodiment, there is provided an orally disintegrable dosage form including alprazolam and in a preferred embodiment, at least one taste masking material (first taste masking material) that can disintegrate in the mouth within about 90 seconds or less and which provides a release of not more than 45% of its content of alprazolam within 5 minutes when tested by a USP 2 apparatus using a USP 2 paddle test as described herein in a media having a pH of 6.8. This can be accomplished with the multi-layered dosage forms described herein or the granulated dosage forms. However, other techniques are also contemplated so long as they provide this same result. In still another embodiment, there is provided an orally disintegrable tablet that can disintegrate in the mouth within about 90 seconds or less and which provides a release of not less than 85% of its content of alprazolam within 5 minutes when tested by a using a USP 2 paddle test as described herein in a media having a pH of 6.0. In still a further embodiment, there is provided an orally disintegrable tablet that can disintegrate in the mouth within about 90 seconds or less and which provides a release of not less than 90% of its content of alprazolam within 5 minutes when tested by a using a USP 2 paddle test as described herein in a media having a pH of 6.0. The pH testing at pH 6 is based on a an average of several tests. This compared with commercial Xanax which exhibited a dissolution under the same conditions of not more than 75% at 5 minutes. See Tables 3-6. Again, this can be accomplished with the multi-layered dosage forms described herein or the granulated dosage forms. However, other techniques are also contemplated so long as they provide this same result.

In a particularly preferred embodiment, there is provided an orally disintegrable tablet that can disintegrate in the mouth within about 90 seconds or less and which provides a release of not more than 35% of its content of alprazolam within 5 minutes in a media having a pH of 6.8, and a release of not less than 90% of its content of alprazolam within 5 minutes when tested by a using a USP 2 paddle test as described herein in a media having a pH of 6.0. Again, this can be accomplished with the multi-layered dosage forms described herein or the granulated dosage forms. However, other techniques are also contemplated so long as they provide this same result.

In another embodiment, the alprazolam-containing layer material, e.g., the combination of the alprazolam and the first taste masking material, can be used as a granulation binder. Granulation can be wet or dry granulation and can be accomplished using any known granulation technique. While it is possible to granulate the alprazolam directly, usually a support or filler, such as microcrystalline cellulose or mannitol, or a combination of fillers and excipients as described herein, may be used in the granulation process with the polymer/alprazolam solution acting as binder/granulation liquid. Sufficient amounts of each ingredient should be used to assure proper particle size distribution and content uniformity. The resulting granulate can be coated with the one or more overcoating layers directly or can first be coated with one or more alprazolam-containing layers prior to application of one or more overcoating layers as described previously for the solid support in the non-granulated aspects of the invention. The resulting granulate and/or coated granulate can next be tabletted or otherwise formed into a dosage form as described herein. Being metered into a capsule or directly compressed into a tablet as a dried granulate are preferred. The relative proportion of the alprazolam and first taste masking material in the granulation is the same as that previously described for the layers sold support embodiments described herein.

### EXAMPLES

### Example 1

Coated Alprazolam 2.57%

| COMPONENT | PRODUCTION | MATERIAL | FOOT |
|---|---|---|---|
| NAME | FORMULA (kg) | FORMULA (mg/g) | NOTES |
| Alprazolam, USP | 5.141 | 25.67 | |
| Sugar Spheres, NF | 143.00 | 714.09 | 1 |
| Eudragit E-100, | 40.4 | 201.7 | |
| EP/JPE | | | |
| Magnesium Stearate, | 11.714 | 58.50 | 2 |
| NF/EP/JP | | | |
| Alcohol, SDA-3A, | 272.6 | N/A | 3 |
| Anhydrous | | | |
| TOTAL | 200.255 | 1000.00 | |

Footnotes:
1 60/80 Grade
2 Non-Bovine grade
3 Alcohol is removed during processing

### Example 2

0.25 mg Alprazolam, ¼″ Tablets

| COMPONENT NAME | QUANTITY (mg/tablet) |
|---|---|
| Alprazolam, Coated¹ | 9.73 |
| Mannitol | 76.07 |
| Disintegrants/binder | 11.00 |
| Magnesium Stearate, NF/EP/JP | 1.50 |
| Natural & Artificial Flavor | 0.75 |
| Sucralose, NF | 0.50 |
| Colloidal Silicon Dioxide, NF/EP | 0.30 |
| Ferric Oxide, NF | 0.15 |
| TOTAL | 100.0 |

Footnotes:
¹Amount based on theoretical potency of 2.57%

### Example 3

0.5 mg Alprazolam, 5/16″ Tablets

| COMPONENT NAME | QUANTITY (mg/tablet) |
|---|---|
| Alprazolam, Coated¹ | 19.46 |
| Mannitol | 152.14 |
| Disintegrants/binder | 22.00 |
| Magnesium Stearate, NF/EP/JP | 3.00 |
| Natural & Artificial Flavor | 1.50 |
| Sucralose, NF | 1.00 |
| Colloidal Silicon Dioxide, NF/EP | 0.60 |
| Ferric Oxide, NF | 0.30 |
| TOTAL | 200.0 |

Footnotes:
¹Amount based on theoretical potency of 2.57%

### Example 4

1.0 mg Alprazolam, 5/16″, Convex Tablets

| COMPONENT NAME | QUANTITY (mg/tablet) |
|---|---|
| Alprazolam, Coated¹ | 38.91 |
| Mannitol | 132.99 |
| Disintegrants/binder | 22.00 |
| Magnesium Stearate, NF/EP/JP | 3.00 |
| Natural & Artificial Flavor | 1.50 |
| Sucralose, NF | 1.00 |
| Colloidal Silicon Dioxide, NF/EP | 0.60 |
| TOTAL | 200.0 |

Footnotes:
¹Amount based on theoretical potency of 2.57%

### Example 5

2.0 mg Alprazolam, ⅜″, Convex Tablets

| COMPONENT NAME | QUANTITY (mg/tablet) |
|---|---|
| Alprazolam, Coated¹ | 77.82 |
| Mannitol | 265.98 |
| Disintegrants/binder | 44.00 |
| Magnesium Stearate, NF/EP/JP | 6.00 |
| Natural & Artificial Flavor | 3.00 |
| Sucralose, NF | 2.00 |
| Colloidal Silicon Dioxide, NF/EP | 1.20 |
| TOTAL | 400.0 |

Footnotes:
¹Amount based on theoretical potency of 2.57%

### Example 6

Dissolution of tablets produced generally in accordance with Examples 1-5 can be tested using a standard USP dissolution apparatus 2 with a paddle speed of 50 rpm, in 250 mL of 70 mM phosphate buffer pH 7.4. The analysis is performed by HPLC. Results

| Tablet | | % Released (minutes) | | | | |
|---|---|---|---|---|---|---|
| strength | # of | 1 | 2 | 3 | 4 | 5 |
| (mg) | tablets/vessel | minute | minutes | minutes | minutes | minutes |
| 0.25 | 8 | 2.54 | 3.96 | 5.23 | 6.23 | 7.23 |
| 0.5 | 4 | 2.49 | 3.87 | 4.94 | 5.81 | 6.57 |
| 1 | 2 | 2.35 | 3.78 | 4.80 | 5.64 | 6.50 |
| 2 | 1 | 2.32 | 3.82 | 5.21 | 6.19 | 6.87 |

Note: These tests reflect batches made of tablets generally falling within the formulations reflected in Examples 2-5. Only one vessel was tested for each strength. This was not the aforementioned test for taste masking. It shows the release profile of tablets in accordance with the present invention other.

### Example 7

0.25 mg Alprazolam Tablet Analysis Release Testing

| Test Method | Claim Specifications | Results | | | |
|---|---|---|---|---|---|
| Physical | Yellow, convex, round | Yellow, convex, round beveled edge scored | | | |
| Appearance | beveled edge scored | tablets. Confirm debossing “SP 321” | | | |
| | tablets. Confirm debossing | on one side and “0.25” on the other. | | | |
| | “SP 321” on one side and | | | | |
| | “0.25” on the other. | | | | |
| Identity by HPLC | Positive for alprazolam | Conforms | | | |
| Assay | 90.0%-110.0% Label Claim | 101.1% | | | |
| Dissolution | Report % Released at 5, 15, | % Released (n = 12) | | | |
| | 30, and 45 min. | 5 min | 15 min | 30 min | 45 min |
| | NLT 80% (Q) at 30 minutes | 96 | 98 | 99 | 99 |
| | | | | Range: | |
| | | | | 91-112 | |

| | | |
|---|---|---|
| Disintegration | Report mean value and the | Avg. = 23 seconds |
| | Range: | Low = 20 High = 30 |
| Water Content | Report Value | 0.48% |
| Hardness | Report Value | 28 N |

Dissolution was tested using the following apparatus and procedure. This testing procedure was also used for examples 8-20. The dissolution of tablets reported in examples 7-20 used tablets produced generally in accordance with examples 1-5. These were not tests of the aforementioned test for taste masking. Parameters:
1. Instrumentation: Dissolution system
Apparatus: USP 2, paddles
Medium: 70 mM potassium phosphate buffer, pH 6.0
Medium Volume: 500 mL
Medium Temperature: 37.0*C*0.5*C
Paddle Speed: 50 rpm
   2. Instrumentation: HPLC system with UV detector
Separation: Reversed-phase, pH 3.0 buffer and acetonitrile
Detection: 254 nm

### Example 8

0.25 mg Alprazolam Tablet Analysis

Release Testing

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 321” on | | | |
| | debossing “SP 321” on one | one side and “0.25” on the other. | | | |
| | side and “0.25” on the other. | | | | |
| Identity by | Positive for alprazolam | Conforms | | | |
| HPLC | | | | | |
| Assay | 90.0%-110.0% Label Claim | 97.8% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 95 | 98 | 98 | 98 |
| | | | | Range: | |
| | | | | 89-106 | |

| | | |
|---|---|---|
| Disintegration | Report mean value and the | Avg. = 21 seconds |
| | Range: | Low = 16 High = 25 |
| Water Content | Report Value | 0.47% |
| Hardness | Report Value | 27 N |

### Example 9

0.5 mg Alprazolam Tablet Analysis

Release Testing

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 322” on | | | |
| | debossing “SP 322” on one | one side and “0.5” on the other. | | | |
| | side and “0.5” on the other. | | | | |
| Identity by | Positive for alprazolam | Conforms | | | |
| HPLC | | | | | |
| Assay | 90.0%-110.0% Label Claim | 101.6% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 96 | 99 | 101 | 101 |
| | | | | Range: | |
| | | | | 90-111 | |

| | | |
|---|---|---|
| Disintegration | Report mean value and the | Avg. = 28 seconds |
| | Range: | Low = 21 High = 36 |
| Water Content | Report Value | 0.47% |
| Hardness | Report Value | 29 N |

### Example 10

0.5 mg Alprazolam Tablet Analysis

Release Testing

| Test Method | Claim Specifications | Results | | | |
|---|---|---|---|---|---|
| Physical | Yellow, convex, round | Yellow, convex, round beveled edge scored | | | |
| Appearance | beveled edge scored | tablets. Confirm debossing “SP 322” on one | | | |
| | tablets. Confirm debossing | side and “0.5” on the other. | | | |
| | “SP 322” on one side and | | | | |
| | “0.5” on the other. | | | | |
| Identity by | Positive for alprazolam | Conforms | | | |
| HPLC | | | | | |
| Assay | 90.0%-110.0% Label Claim | 99.6% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 95 | 100 | 100 | 100 |
| | | | | Range: | |
| | | | | 93-108 | |

| | | |
|---|---|---|
| Disintegration | Report mean value and the | Avg. = 25 seconds |
| | Range: | Low = 18 High = 35 |
| Water Content | Report Value | 0.45% |
| Hardness | Report Value | 30 N |

### Example 11

0.25 mg Alprazolam Tablet Analysis

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 321” on | | | |
| | debossing “SP 321” on one | one side and “0.25” on the other. | | | |
| | side and “0.25” on the other. | | | | |
| Identity by | Positive for alprazolam | Conforms | | | |
| HPLC | | | | | |
| Assay | 90.0%-110.0% Label Claim | 101.1% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 96 | 98 | 99 | 99 |
| | | | | Range: | |
| | | | | 91-112 | |

| | | |
|---|---|---|
| Disintegration | Report mean value and the | Avg. = 23 seconds |
| | Range: | Low = 20 High = 30 |
| Water Content | Report Value | 0.56% |
| Hardness | Report Value | 28 N |

### Example 12

0.25 mg Alprazolam Tablet Analysis

Release Testing

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 321” on | | | |
| | debossing “SP 321” on one | one side and “0.25” on the other. | | | |
| | side and “0.25” on the other. | | | | |
| Identity by | Positive for alprazolam | Conforms | | | |
| HPLC | | | | | |
| Assay | 90.0%-110.0% Label Claim | 97.8% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 95 | 98 | 98 | 98 |
| | | | | Range: | |
| | | | | 89-106 | |

| | | |
|---|---|---|
| Disintegration | Report mean value and the | Avg. = 21 seconds |
| | Range: | Low = 16 High = 25 |
| Water Content | Report Value | 0.56% |
| Hardness | Report Value | 27 N |

### Example 13

0.5 mg Alprazolam Tablet Analysis

Release Testing

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 322” on | | | |
| | debossing “SP 322” on one | one side and “0.5” on the other. | | | |
| | side and “0.5” on the other. | | | | |
| Identity by | Positive for alprazolam | Conforms | | | |
| HPLC | | | | | |
| Assay | 90.0%-110.0% Label Claim | 101.6% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 96 | 99 | 101 | 101 |
| | | | | Range: | |
| | | | | 90-111 | |

| | | |
|---|---|---|
| Disintegration | Report mean value and the | Avg. = 28 seconds |
| | Range: | Low = 21 High = 36 |
| Water Content | Report Value | 0.63% |
| Hardness | Report Value | 29 N |

### Example 14

0.5 mg Alprazolam Tablet Analysis

Release Testing

| Test Method | Claim Specifications | Results | | | |
|---|---|---|---|---|---|
| Physical | Yellow, convex, round beveled | Yellow, convex, round beveled edge scored | | | |
| Appearance | edge scored tablets. Confirm | tablets. Confirm debossing “SP 322” on | | | |
| | debossing “SP 322” on one | one side and “0.5” on the other. | | | |
| | side and “0.5” on the other. | | | | |
| Identity by | Positive for alprazolam | Conforms | | | |
| HPLC | | | | | |
| Assay | 90.0%-110.0% Label Claim | 99.6% | | | |
| Dissolution | Report % Released at 5, 15, | | | | |
| | 30, and 45 min. | % Released (n = 12) | | | |
| | NLT 80% (Q) at 30 minutes | 5 min | 15 min | 30 min | 45 min |
| | | 95 | 100 | 100 | 100 |
| | | | | Range: | |
| | | | | 93-108 | |

| | | |
|---|---|---|
| Disintegration | Report mean value and the | Avg. = 25 seconds |
| | Range: | Low = 18 High = 35 |
| Water Content | Report Value | 0.61% |
| Hardness | Report Value | 30 N |

### Example 15

1 mg Alprazolam Tablet Analysis

Release Testing

| Test Method | Claim Specifications | Results | | | |
|---|---|---|---|---|---|
| Physical | White, convex, round beveled | White, convex, round beveled edge scored | | | |
| Appearance | edge scored tablets. Confirm | tablets. Confirm debossing “SP 323” on one | | | |
| | debossing “SP 323” on one | side and “1” on the other. | | | |
| | side and “1” on the other. | | | | |
| Identity by | Positive for alprazolam | Conforms | | | |
| HPLC | | | | | |
| Assay | 90.0%-110.0% Label Claim | 98.7% | | | |
| Dissolution | Report % Released at 5, 15, | % Released (n = 12) | | | |
| | 30, and 45 min. | 5 min | 15 min | 30 min | 45 min |
| | NLT 80% (Q) at 30 minutes | 92 | 98 | 99 | 99 |
| | | | | Range: | |
| | | | | 95-105 | |

| | | |
|---|---|---|
| Disintegration | Report mean value and the | Avg. = 25 seconds |
| | Range: | Low = 20 High = 31 |
| Water Content | Report Value | 0.62% |
| Hardness | Report Value | 30 N |

### Example 16

1 mg Alprazolam Tablet Analysis

Release Testing

| Test Method | Claim Specifications | Results | | | |
|---|---|---|---|---|---|
| Physical | White, convex, round beveled | White, convex, round beveled edge scored | | | |
| Appearance | edge scored tablets. Confirm | tablets. Confirm debossing “SP 323” on one | | | |
| | debossing “SP 323” on one | side and “1” on the other. | | | |
| | side and “1” on the other. | | | | |
| Identity by | Positive for alprazolam | Conforms | | | |
| HPLC | | | | | |
| Assay | 90.0%-110.0% Label Claim | 100.7% | | | |
| Dissolution | Report % Released at 5, 15, | % Released (n = 12) | | | |
| | 30, and 45 min. | 5 min | 15 min | 30 min | 45 min |
| | NLT 80% (Q) at 30 minutes | 94 | 98 | 99 | 99 |
| | | | | Range: | |
| | | | | 94-107 | |

| | | |
|---|---|---|
| Disintegration | Report mean value and the | Avg. = 26 seconds |
| | Range: | Low = 21 High = 35 |
| Water Content | Report Value | 0.64% |
| Hardness | Report Value | 31 N |

### Example 17

2 mg Alprazolam Tablet Analysis

Release Testing

| Test Method | Claim Specifications | Results | | | |
|---|---|---|---|---|---|
| Physical | White, convex, round beveled | White, convex, round beveled edge scored | | | |
| Appearance | edge scored tablets. Confirm | tablets. Confirm debossing “SP 324” on one | | | |
| | debossing “SP 324” on one | side and “2” on the other. | | | |
| | side and “2” on the other. | | | | |
| Identity by | Positive for alprazolam | Conforms | | | |
| HPLC | | | | | |
| Assay | 90.0%-110.0% Label Claim | 99.7% | | | |
| Dissolution | Report % Released at 5, 15, | % Released (n = 12) | | | |
| | 30, and 45 min. | 5 min | 15 min | 30 min | 45 min |
| | NLT 80% (Q) at 30 minutes | 94 | 99 | 99 | 100 |
| | | | | Range: | |
| | | | | 96-107 | |

| | | |
|---|---|---|
| Disintegration | Report mean value and the | Avg. = 35 seconds |
| | Range: | Low = 26 High = 40 |
| Water Content | Report Value | 0.60% |
| Hardness | Report Value | 35 N |

### Example 18

2 mg Alprazolam Tablet Analysis

Release Testing

| Test Method | Claim Specifications | Results | | | |
|---|---|---|---|---|---|
| Physical | White, convex, round | White, convex, round beveled edge scored | | | |
| Appearance | beveled edge scored | tablets. Confirm debossing “SP 324” on one | | | |
| | tablets. Confirm debossing | side and “2” on the other. | | | |
| | “SP 324” on one side and | | | | |
| | “2” on the other. | | | | |
| Identity by HPLC | Positive for alprazolam | Conforms | | | |
| Assay | 90.0%-110.0% Label Claim | 101.9% | | | |
| Dissolution | Report % Released at 5, 15, 30, and | % Released (n = 12) | | | |
| | 45 min. NLT 80% (Q) at 30 minutes | 5 min | 15 min | 30 min | 45 min |
| | | 92 | 101 | 101 | 102 |
| | | | | Range: | |
| | | | | 97-109 | |

| | | |
|---|---|---|
| Disintegration | Report mean value and the | Avg. = 35 seconds |
| | Range: | Low = 31 High = 41 |
| Water Content | Report Value | 0.58% |
| Hardness | Report Value | 36 N |

### Example 19

0.5 mg Alprazolam Flat Faced Tablet Analysis

Testing

| Test Method | Claim Specifications | Results | |
|---|---|---|---|
| Physical Appearance | Yellow, flat faced, round | Complies | |
| | beveled edge scored | | |
| | tablets. Confirm debossing | | |
| | “SP 322” on one side and | | |
| | “0.5” on the other. | | |
| Identity by HPLC | Positive for alprazolam | Complies | |
| Assay | 90.0%-110.0% Label Claim | 101.3% label claim | |
| Dissolution | (Q) = 80% at 30 minutes | Avg. (30 min) = 102% Released | |
| | 1) NMT 0 less than 85% | Vessel | (%) |
| | (n = 6) | 1 | 99 |
| | 2) Average NLT 80%; | 2 | 99 |
| | NMT 0 < 65% (N = 12) | 3 | 106 |
| | 3) Average NLT 80%; | 4 | 108 |
| | NMT 2 < 65%, and | 5 | 104 |
| | NMT 0 < 55% (n = 24) | 6 | 102 |
| | | 7 | 108 |
| | | 8 | 103 |
| | | 9 | 92 |
| | | 10 | 102 |
| | | 11 | 104 |
| | | 12 | 99 |

| Avg. (10 min) = 101% Released⁵ | |
|---|---|
| Vessel | (%) |
| 1 | 97 |
| 2 | 96 |
| 3 | 105 |
| 4 | 107 |
| 5 | 103 |
| 6 | 101 |
| 7 | 106 |
| 8 | 101 |
| 9 | 91 |
| 10 | 100 |
| 11 | 102 |
| 12 | 97 |

| | | |
|---|---|---|
| Water Content | NMT 2.5% | 0.65% |
| Disintegration | Average NMT 60 seconds | Average (n = 18) = 28 seconds |
| Time | | |

⁵The 10 minute dissolution results are for information only.

### Example 20

0.5 mg Alprazolam Flat Faced Tablet Analysis

Testing

| Test Method | Claim Specifications | Results | |
|---|---|---|---|
| Physical Appearance | Yellow, flat faced, round | Complies | |
| | beveled edge scored | | |
| | tablets. Confirm debossing | | |
| | “SP 321” on one side and | | |
| | “0.25” on the other. | | |
| Identity by HPLC | Positive for alprazolam | Complies | |
| Assay | 90.0%-110.0% Label Claim | 101.6% label claim | |
| Dissolution | (Q) = 80% at 30 minutes | Avg. (30 min) = 102% Released | |
| | 1) NMT 0 less than 85% | Vessel | (%) |
| | (n = 6) | 1 | 93 |
| | 2) Average NLT 80%; | 2 | 101 |
| | NMT 0 < 65% (N = 12) | 3 | 113 |
| | 3) Average NLT 80%; | 4 | 104 |
| | NMT 2 < 65%, and | 5 | 109 |
| | NMT 0 < 55% (n = 24) | 6 | 100 |
| | | 7 | 96 |
| | | 8 | 104 |
| | | 9 | 97 |
| | | 10 | 103 |
| | | 11 | 102 |
| | | 12 | 101 |

| Avg. (10 min) = 101% Released⁶ | |
|---|---|
| Vessel | (%) |
| 1 | 92 |
| 2 | 100 |
| 3 | 112 |
| 4 | 102 |
| 5 | 109 |
| 6 | 100 |
| 7 | 96 |
| 8 | 104 |
| 9 | 95 |
| 10 | 102 |
| 11 | 100 |
| 12 | 101 |

| | | |
|---|---|---|
| Water Content | NMT 2.5% | 0.68% |
| Disintegration | Average NMT 60 seconds | Average (n = 18) = 36 seconds |
| Time | | |

⁶The 10 minute dissolution results are for information only.

### Example 21

2.5 mg/g Batch Manufacturing

This process is used with the formulation described in examples 1 and 2.

The same process may be used generally for all of the formulations of examples 1-5.

Ethanol is used to dissolve the Eudragit E-100 which is mixed until dissolution is complete. Magnesium stearate is mixed in until homogeneous followed by addition of alprazolam. Mixing continues until homogenous. Sucrose spheres are charged to a Wurtzer fluid bed reactor and coated with the homogenous mixture including alprazolam to form the alprazolam-containing layer. For the overcoating layer, ethanol is used to dissolve the Eudragit E-100 which is mixed until dissolution is complete. Magnesium stearate is mixed in until homogeneous. This is coated over the alprazolam-containing coating on the sugar spheres using the same reactor, without emptying the reactor. The particles are screened and mixed with the other materials, then the lubricant is added.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A taste masked formulation comprising: a solid support, at least one alprazolam-containing layer covering at least a portion of said solid support, at least one overcoating layer covering at least a portion of said alprazolam-containing layer, and at least one additional ingredient, said alprazolam-containing layer comprising alprazolam and at least one first taste masking material.

2. The taste masked formulation of claim 1, wherein said at least one additional ingredient is selected from the group consisting of binders, glidants, disintegrants, effervescent couples, colors, flavors, coatings, lubricants and carriers.

3. The taste masked formulation of claim 1, wherein said solid support is a particle, crystal, granule, capsule, microparticle, microgranule, microcrystal, or microcapsule.

4. The taste masked formulation of claim 3, wherein said solid support comprises sugar.

5. The taste masked formulation of claim 1, wherein said solid support has an average particle size between about 10 microns and about 1,000 microns.

6. The taste masked formulation of claim 5, wherein said solid support has an average particle size between about 20 microns and about 600 microns.

7. The taste masked formulation of claim 1, wherein said overcoating layer comprises at least one second taste masking material and said first and said second taste masking materials are the same material.

8. The taste masked formulation of claim 7, wherein said first and said second taste masking materials are Eudragit E-100.

9. The taste masked formulation of claim 1, wherein said at least one alprazolam-containing layer covers substantially all of said solid support, and said at least one overcoating layer covers substantially all of said alprazolam-containing layer.

10. The taste masked formulation of claim 1, wherein said alprazolam-containing layer is present on said solid support in an amount of at least about 85% by weight of said alprazolam and said first taste masking material used in coating said solid support.

11. The taste masked formulation of claim 1, wherein said overcoating layer is present in an amount of at least about 85% by weight of the material used in coating said alprazolam-containing layer coated solid support.

12. The taste masked formulation of claim 1, wherein said first taste masking material is pH dependent and becomes soluble at a pH of about 6.5 or less.

13. The taste masked formulation of claim 1, wherein the overcoating layer comprises a second taste masking material which is pH dependent and becomes soluble at a pH of about 6.5 or less.

14. The taste masked formulation of claim 1, wherein said overcoating layer is substantially free of said alprazolam.

15. The taste masked formulation of claim 1, wherein said alprazolam is provided in an amount of between about 0.1 micrograms and about 2 grams.

16. The taste masked formulation of claim 15, wherein said alprazolam is provided in an amount of between about 10 micrograms and about 0.5 grams.

17. The taste masked formulation of claim 1, further comprising a plurality of alprazolam-containing layers wherein at least one alprazolam-containing layer comprises alprazolam and at least one first taste masking material.

18. The taste masked formulation of claim 1, further comprising a plurality of overcoating layers.

19. The taste masked formulation of claim 1, wherein said overcoating layer comprising at least one second taste masking material

20. A pharmaceutical dosage form comprising: alprazolam and at least a first taste masking material wherein said dosage form disintegrates in the mouth within about 90 seconds or less and provides a release of not more than about 45% of its content of alprazolam within about 5 minutes when tested by a USP 2 paddle test in a media having a pH of about 6.8 or provides a release of not less than about 85% of its content of alprazolam within about 5 minutes when tested by a USP 2 paddle test in a media having a pH of about 6.0, and wherein said at least one first taste masking material is pH dependent and become soluble at a pH of about 6.5 or less.

21. The pharmaceutical dosage form of claim 20, further comprising: a solid support, at least one alprazolam-containing layer covering at least a portion of said solid support, at least one overcoating layer covering at least a portion of said alprazolam-containing layer, said alprazolam-containing layer comprising alprazolam and said at least one first taste masking material and said overcoating layer comprising at least one second taste masking material and at least one additional ingredient selected from the group consisting of binders, glidants, disintegrants, effervescent coupes, colors, flavors, coatings, lubricants and carriers, said pharmaceutical dosage form being in the form of a tablet, capsule, caplet, gel cap, powder, gum, film, liquid, syrup, or suspension.

22. The pharmaceutical dosage form of claim 21, wherein said solid support is a particle, crystal, granule, capsule, microparticle, microgranule, microcrystal, or microcapsule.

23. The pharmaceutical dosage form of claim 22, wherein said solid support comprises sugar.

24. The pharmaceutical dosage form of claim 21, wherein said solid support has an average particle size between about 10 microns and about 1,000 microns.

25. The pharmaceutical dosage form of claim 24, wherein said solid support has an average particle size between about 20 microns and about 600 microns.

26. The pharmaceutical dosage form of claim 21, wherein said first and said second taste masking materials are the same material.

27. The pharmaceutical dosage form of claim 26, wherein said first and said second taste masking materials are Eudragit E-100.

28. The pharmaceutical dosage form of claim 21, wherein said alprazolam is provided in an amount of between about 0.1 micrograms and about 2 grams.

29. The pharmaceutical dosage form of claim 28, wherein said alprazolam is provided in an amount of between about 10 micrograms and about 0.5 grams.

30. The pharmaceutical dosage form of claim 20, further comprising at least one additional ingredient selected from the group consisting of binders, glidants, disintegrants, effervescent coupes, colors, flavors, coatings, lubricants and carriers, said pharmaceutical dosage form being in the form of a tablet, capsule, caplet, gel cap, powder, gum, film, liquid, syrup, or suspension and wherein said alprazolam and said first taste masking material were granulated.

31. A method of treating anxiety disorders, panic disorders, agoraphobia and/or irritable bowel syndrome in a subject in need thereof comprising the steps of administering to said subject an orally disintegrable tablet comprising: at least one solid support, at least one alprazolam-containing layer covering at least a portion of said at least one solid support, and at least one overcoating layer covering at least a portion of said at least one alprazolam-containing layer, said at least one alprazolam-containing layer comprising alprazolam and at least one first taste masking material and said overcoating layer comprising at least one second taste masking material and at least one additional ingredient selected from the group consisting of binders, glidants, effervescent couples, colors, flavors, coatings, lubricants and carriers, said orally disintegrable tablet being in the form of a compressed tablet which can disintegrate in the mouth of a patient within about 60 seconds, placing said orally disintegrable tablet into the mouth of said subject, maintaining said tablet in said mouth for a time which is sufficient to allow the tablet to disintegrate and/or dissolve, and swallowing the resulting disintegrated and/or dissolved tablet.

32. The method of claim 31, wherein said first and said second taste masking materials are the same material.

33. The method of claim 32, wherein said first and said second taste masking materials are Eudragit E-100.

34. The method of claim 31, wherein said first taste masking material is pH dependent and becomes soluble at a pH of about 6.5 or less.

35. The method of claim 31, wherein said second taste masking material is pH dependent and becomes soluble at a pH of about 6.5 or less.

36. The method of claim 31, wherein said alprazolam is provided in an amount of between about 0.1 micrograms and about 2 grams.

37. The method of claim 36, wherein said alprazolam is provided in an amount of between about 10 micrograms and about 0.5 grams.

38. The method of claim 31, wherein said orally disintegrable tablet disintegrates in less than about 45 seconds in said mouth of said subject.

39. A pharmaceutical dosage form comprising: alprazolam and at least a first taste masking material wherein said orally disintegrable dosage form can disintegrate in the mouth within about 90 seconds or less and provides a release of not more than about 45% of its content of alprazolam within about 5 minutes when tested by a USP 2 paddle test in a media having a pH of about 6.8 and provides a release of not less than about 85% of its content of alprazolam within about 5 minutes when tested by a USP 2 paddle test in a media having a pH of about 6.0.

40. The pharmaceutical dosage form of claim 39, wherein said at least one additional ingredient is selected from the group consisting of binders, glidants, disintegrants, effervescent couples, colors, flavors, coatings, lubricants and carriers.

41. The taste masked formulation of claim 40, wherein said first and said second taste masking materials are Eudragit E-100.

42. The pharmaceutical dosage form of claim 20, wherein said alprazolam is present in an amount of about 0.25 mg and said dosage form provides release of not more than about 20% of its contents of alprazolam within about 5 minutes when tested by a USP 2 paddle test in a media having a pH of about 6.8.

43. The pharmaceutical dosage form of claim 20, wherein said alprazolam is present in an amount of about 0.5 mg or more and said dosage form provides a release of not more than about 25% of its contents of alprazolam within about 5 minutes when tested by a USP 2 paddle test in a media having a pH of about 6.8.

44. The pharmaceutical dosage form of claim 39, wherein said alprazolam is present in an amount of about 0.25 mg and said dosage form provides release of not more than about 20% of its contents of alprazolam within about 5 minutes when tested by a USP 2 paddle test in a media having a pH of about 6.8.

45. The pharmaceutical dosage form of claim 39, wherein said alprazolam is present in an amount of about 0.5 mg or more and said dosage form provides a release of not more than about 25% of its contents of alprazolam within about 5 minutes when tested by a USP 2 paddle test in a media having a pH of about 6.8.
